(19) United States
(12) Patent Application Publication
(10) Pub. No.: US 2015/0299800 A1
(43) Pub. Date: Oct. 22, 2015

(54) MONITORING MANTLE CELL LYMPHOMA CLONOTYPES IN PERIPHERAL BLOOD AFTER IMMUNOTRANSPLANT

(71) Applicant: SEQUENTA, INC., South San Francisco, CA (US)

(72) Inventors: Malek FAHAM, South San Francisco, CA (US); Victoria CARLTON, South San Francisco, CA (US)

(21) Appl. No.: 14/437,470

(22) PCT Filed: Oct. 18, 2013
(86) PCT No.: PCT/US2013/065757

§ 371 Date: Apr. 21, 2015

Related U.S. Application Data
(60) Provisional application No. 61/716,995, filed on Oct. 22, 2012.

(51) Int. Cl. C12Q 1/68

(52) U.S. Cl. CPC C12Q 1/6886; C12Q 2600/156; C12Q 2600/158

(57) ABSTRACT

The invention is directed to a method of monitoring a mantle cell lymphoma residual disease in an immunotransplant patient by post-treatment analysis of clonotype profiles from patient blood samples. In some embodiments, methods of the invention comprising steps of (a) treating a patient by immunotransplanting the patient with vaccine-primed autologous T cells; (b) obtaining a peripheral blood sample from the patient comprising B-cells and/or cell-free nucleic acids; (c) amplifying molecules of nucleic acid from the B-cells of the sample and/or cell-free nucleic acids in the sample, the molecules of nucleic acid comprising recombined DNA sequences from immunoglobulin genes; (d) sequencing the amplified molecules of nucleic acid to form a clonotype profile; and (e) determining from the clonotype profile a presence, absence and/or level of the one or more patient-specific clonotypes correlated with the mantle cell lymphoma, including phylogenic clonotypes thereof.

### CROSS-REFERENCE

This application claims the benefit of U.S. Provisional Patent Application No. 61/716,995, filed Oct. 22, 2012, which application is herein incorporated by reference in its entirety.

### BACKGROUND OF THE INVENTION

Mantle cell lymphoma (MCL) has a poor long-term prognosis. Though autologous transplant prolongs survival, novel and mechanistically distinct therapies are needed to target residual, myeloablation-resistant tumor cells that result in relapse. Trials of CpG-based vaccines for low-grade lymphoma have shown induction of anti-tumor T cells and clinical responses, e.g. Brody et al, J. Clin. Oncol., 28(28): 4324-4332 (2010). A promising treatment approach, referred to as “immunotransplant” or “immunotransplanting,” comprises the following steps: 1) CpG-based vaccination, 2) harvest of vaccine-primed T cells, 3) myeloablation with stem cell rescue, and 4) T cell re-infusion, e.g. Brody et al, Blood, 113(1): 85-94 (2009). Immunotransplant amplifies the proportion of anti-tumor T cells by an order of magnitude and is capable of curing even bulky, systemic lymphoma burden. Maintaining immunotransplant patients in a disease-free state depends on the ability to monitor the occurrence of minimal residual disease (MRD) with high sensitivity.

Profiles of nucleic acids encoding immune molecules, such as T cell or B cell receptors, or their components, contain a wealth of information on the state of health or disease of an organism, so that the use of such profiles as diagnostic or prognostic indicators has been proposed for a wide variety of conditions, e.g. Faham and Willis, U.S. patent publication 2010/0151471 and 2011/0207134; Freeman et al, Genome Research, 19: 1817-1824 (2009); Boyd et al, Sci. Transl. Med., 1(12): 12ra23 (2009); He et al, Oncotarget (Mar. 8, 2011). Such sequence-based profiles are capable of much greater sensitivity than other approaches for measuring immune repertoires or their component clonotypes, e.g. van Dongen et al, Leukemia, 17: 2257-2317 (2003); Ottensmeier et al, Blood, 91: 4292-4299 (1998).

It would be advantageous for MCL patients if a more sensitive and convenient assay was available to monitor and detect reliably minimal residual disease using sequence-based immune repertoire analysis.

### SUMMARY OF THE INVENTION

The present invention is directed to methods for monitoring MCL patients for minimal residual disease using sequence-based immune repertoire analysis of peripheral blood samples. The invention is exemplified in a number of implementations and applications, some of which are summarized below and throughout the specification.

In one aspect the invention is directed to a method of monitoring a mantle cell lymphoma residual disease in a patient by one or more patient-specific clonotypes correlated with the mantle cell lymphoma, wherein the method comprises the following steps: (a) treating a patient by immunotransplanting the patient with vaccine-primed autologous T cells; (b) obtaining a peripheral blood sample from the patient comprising B-cells and/or cell-free nucleic acids from B-cells; (c) amplifying molecules of nucleic acid from the B-cells of the sample and/or cell-free nucleic acids from B-cells in the sample, the molecules of nucleic acid comprising recombined DNA sequences from immunoglobulin genes; (d) sequencing the amplified molecules of nucleic acid to form a clonotype profile; and (e) determining from the clonotype profile a presence, absence and/or level of the one or more patient-specific clonotypes correlated with the mantle cell lymphoma, including previously unrecorded phylogenic clonotypes thereof.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the invention.

### BRIEF DESCRIPTION OF THE DRAWINGS

The novel features of the invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIGS. 1A-1C show a two-staged PCR scheme for amplifying and sequencing immunoglobulin genes.

FIG. 2A illustrates details of one embodiment of determining a nucleotide sequence of the PCR product of FIG. 1C. FIG. 2B illustrates details of another embodiment of determining a nucleotide sequence of the PCR product of FIG. 1C.

FIG. 3A illustrates a PCR scheme for generating three sequencing templates from an IgH chain in a single reaction. FIGS. 3B-3C illustrate a PCR scheme for generating three sequencing templates from an IgH chain in three separate reactions after which the resulting amplicons are combined for a secondary PCR to add P5 and P7 primer binding sites. FIG. 3D illustrates the locations of sequence reads generated for an IgH chain.

### DETAILED DESCRIPTION OF THE INVENTION

The practice of the present invention may employ, unless otherwise indicated, conventional techniques and descriptions of molecular biology (including recombinant techniques), bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; and Molecular Cloning: A Laboratory Manual (all from Cold Spring Harbor Laboratory Press); and the like.

In most lymphoid neoplasms, such as mantle cell lymphoma, disease cells express clonotypes that are correlated with the disease state. That is, the level of clonotypes produced by the disease cells within a clonotype profile is monotonically related to the state of the disease. For lymphoid neoplasms, the level of correlating clonotype typically relates directly to the extent, or negative prognosis, of the disease. In some instances, this relationship may be expressed as a number using a clonality measure of a clonotype profile. That is, a clonality measure converts a complex data set (the clonotype profile) to a single number, the value of which is a measure of how a clonotype profile is dominated by one or a few clonotypes. Typically, the greater the clonality of a clonotype profile, the worse the disease state. Correlating clonotypes of mantle cell lymphoma may be determined by a variety of techniques. In one approach, one or more correlating clonotypes are identified at diagnosis as the predominant clonotype or clonotypes in a clonotype profile, e.g. from a sample of bone marrow, lymph node tissue, or the like. In part, the present invention is the recognition and appreciation that mantle cell lymphoma disease status can be determined and/or monitored by determining the level of a disease correlating clonotype in a clonotype profile derived from a peripheral blood sample. In particular, the presence of correlating clonotypes in the peripheral blood of a mantle cell lymphoma patient (either from intact cells or from cell-free nucleic acids, such as cell-free DNA) indicates a worse prognosis for disease progression than if no correlating clonotypes are detected.

In further embodiments, methods of the invention comprise the following steps: (a) obtaining a peripheral blood sample from the patient, the sample comprising B-cells and/or cell-free nucleic acids; (b) extracting a nucleic acid sample from the peripheral blood sample; (c) amplifying in a polymerase chain reaction nucleic acid molecules from the nucleic acid sample, the nucleic acid molecules encoding a VDJ region of an IgH or a portion thereof; (c) sequencing the amplified nucleic acid molecules to form a clonotype profile; and (d) determining from the clonotype profile a level of each of the one or more patient-specific clonotypes correlated with the DLBCL, wherein such levels include phylogenic clonotypes of each of such one or more patient-specific clonotypes.

In some embodiments, the step of sequencing includes generating sequence reads in a range of from 20 to 400 nucleotides for determining a sequence of each clonotype.

In some embodiments, methods of the invention further include a step of treating a patient by transplanting bone marrow based on the level of the one or more patient-specific clonotypes or phylogenic clonotypes thereof that are measured. In some of these embodiments, the step of transplanting is implemented whenever the level of said one or more patient-specific clonotypes and phylogenic clonotypes thereof exceeds fifty percent of a level of the one or more patient-specific clonotypes in a diagnostic sample.

In some embodiments, each of the clonotype profiles from the peripheral blood sample includes every clonotype present at a frequency of 0.1 percent or greater with a probability of ninety-nine percent; or at a frequency of 0.01 percent or greater with a probability of ninety-nine percent; or at a frequency of 0.001 percent or greater with a probability of ninety-nine percent; or at a frequency of 0.0001 percent or greater with a probability of ninety-nine percent.

In some embodiments, the step of amplifying includes amplifying nucleic acid molecules from a peripheral blood sample comprising at least 10⁵ B cells; or at least 10⁶ B cells; or at least 10⁷ B cells; or at least 10⁸ B cells.

### Samples

Clonotype profiles for methods of the invention are generated from patient samples, which may be from a tumor or peripheral blood in the case of diagnostic samples or which are from peripheral blood in the case of samples for monitoring residual disease. One or more clonotypes correlated with a DLBCL are determined from a diagnostic sample. Usually the one or more clonotypes correlated with a DLBCL are those present in a clonotype profile with the highest frequencies. In some cases, there may be a single correlated clonotype and in other cases there may be multiple clonotypes correlated with a DLBCL. Tumor samples may be taken from any tissue affected by a DLBCL, which includes lymph nodes or outside of the lymphatic system, the gastrointestinal tract, testes, thyroid, skin, breast, bone, or brain. As mentioned above, clonotype profiles for monitoring residual disease are generated from a sample of nucleic acids extracted from peripheral blood. The nucleic acids of the sample may from B-cells from a cell-containing fraction of the peripheral blood or from a cell free fraction of the peripheral blood, such as plasma or serum. In one embodiment, a sample includes at least 1,000 B cells; but more typically, a sample includes at least 10,000 B cells, and more typically, at least 100,000 B cells. In another aspect, a sample includes a number of B cells in the range of from 1000 to 1,000,000 B cells. Adequate sampling of the cells is an important aspect of interpreting the repertoire data, as described further below in the definitions of “clonotype” and “repertoire.” The number of cells in a sample sets a limit on the sensitivity of a measurement. For example, in a sample containing 1,000 B cells, the lowest frequency of clonotype detectable is 1/1000 or 0.001, regardless of how many sequencing reads are obtained when the DNA of such cells is analyzed by sequencing.

A sample for use with the invention can include DNA (e.g., genomic DNA) or RNA (e.g., messenger RNA). The nucleic acid can be cell-free DNA or RNA, e.g. extracted from the circulatory system, Vlassov et al, Curr. Mol. Med., 10: 142-165 (2010); Swamp et al, FEBS Lett., 581: 795-799 (2007). In the methods of the provided invention, the amount of RNA or DNA from a subject that can be analyzed includes, for example, as low as a single cell in some applications (e.g., a calibration test with other cell selection criteria, e.g. morphological criteria) and as many as 10 million of cells or more, which translates into a quantity of DNA in the range of from 6 pg-60 ug, and a quantity of RNA in the range of from 1 pg-10 ug. In some embodiments, a nucleic acid sample is a DNA sample of from 6 pg to 60 ug. In other embodiments, a nucleic acid sample is a DNA sample from 100 μL to 10 mL of peripheral blood; in other embodiments, a nucleic acid sample is a DNA sample from a cell free fraction of from 100 μL to 10 mL of peripheral blood.

In some embodiments, a sample of lymphocytes or cell free nucleic acid is sufficiently large so that substantially every B cell with a distinct clonotype is represented therein, thereby forming a “repertoire” of clonotypes. In one embodiment, to achieve substantial representation of every distinct clonotype, a sample is taken that contains with a probability of ninety-nine percent every clonotype of a population present at a frequency of 0.001 percent or greater. In another embodiment, a sample is taken that contains with a probability of ninety-nine percent every clonotype of a population present at a frequency of 0.0001 percent or greater. And in another embodiment, a sample is taken that contains with a probability of ninety-nine percent every clonotype of a population present at a frequency of 0.00001 percent or greater. In one embodiment, a sample of B cells includes at least one half million cells, and in another embodiment such sample includes at least one million cells.

Nucleic acid samples may be obtained from peripheral blood using conventional techniques, e.g. Innis et al, editors, PCR Protocols (Academic Press, 1990); or the like. For example, white blood cells may be separated from blood samples using convention techniques, e.g. RosetteSep kit (Stem Cell Technologies, Vancouver, Canada). Blood samples may range in volume from 100 μL to 10 mL; in one aspect, blood sample volumes are in the range of from 100 μL to 2 mL. DNA and/or RNA may then be extracted from such blood sample using conventional techniques for use in methods of the invention, e.g. DNeasy Blood & Tissue Kit (Qiagen, Valencia, Calif.). Optionally, subsets of white blood cells, e.g. lymphocytes, may be further isolated using conventional techniques, e.g. fluorescently activated cell sorting (FACS) (Becton Dickinson, San Jose, Calif.), magnetically activated cell sorting (MACS) (Miltenyi Biotec, Auburn, Calif.), or the like. For example, memory B cells may be isolated by way of surface markers CD19 and CD27.

Cell-free DNA may also be extracted from peripheral blood samples using conventional techniques, e.g. Lo et al, U.S. Pat. No. 6,258,540; Huang et al, Methods Mol. Biol., 444: 203-208 (2008); and the like, which are incorporated herein by reference. By way of nonlimiting example, peripheral blood may be collected in EDTA tubes, after which it may be fractionated into plasma, white blood cell, and red blood cell components by centrifugation. DNA from the cell free plasma fraction (e.g. from 0.5 to 2.0 mL) may be extracted using a QIAamp DNA Blood Mini Kit (Qiagen, Valencia, Calif.), or like kit, in accordance with the manufacturer's protocol.

Since identifying recombinations are present in the DNA of each individual's adaptive immunity cell as well as their associated RNA transcripts, either RNA or DNA can be sequenced in the methods of the provided invention. A recombined sequence from a B-cell encoding an immunoglobulin molecule, or a portion thereof, is referred to as a clonotype. The DNA or RNA can correspond to sequences from immunoglobulin (Ig) genes that encode antibodies.

The DNA and RNA analyzed in the methods of the invention correspond to sequences encoding heavy chain immunoglobulins (IgH). Each chain is composed of a constant (C) and a variable region. For the heavy chain, the variable region is composed of a variable (V), diversity (D), and joining (J) segments. Several distinct sequences coding for each type of these segments are present in the genome. A specific VDJ recombination event occurs during the development of a B-cell, marking that cell to generate a specific heavy chain. Somatic mutation often occurs close to the site of the recombination, causing the addition or deletion of several nucleotides, further increasing the diversity of heavy chains generated by B-cells. The possible diversity of the antibodies generated by a B-cell is then the product of the different heavy and light chains. The variable regions of the heavy and light chains contribute to form the antigen recognition (or binding) region or site. Added to this diversity is a process of somatic hypermutation which can occur after a specific response is mounted against some epitope.

In accordance with the invention, primers may be selected to generate amplicons of recombined nucleic acids extracted from B lymphocytes. Such sequences may be referred to herein as “somatically rearranged regions,” or “somatically recombined regions,” or “recombined sequences.” Somatically rearranged regions may comprise nucleic acids from developing or from fully developed lymphocytes, where developing lymphocytes are cells in which rearrangement of immune genes has not been completed to form molecules having full V(D)J regions. Exemplary incomplete somatically rearranged regions include incomplete IgH molecules (such as, molecules containing only D-J regions).

### Depletion of Granulocytes for Enhanced Sensitivity

In some embodiments, sensitivity of DLBCL cell detection in peripheral blood may be enhanced by removing non-lymphocytes from a sample prior to nucleic acid extraction. A significant non-lymphocyte component of peripheral blood consists of granulocytes, which comprise neutrophils, basophils and eosinophils. In one embodiment, a step of removing granulocytes from a sample of peripheral blood may be carried out in a reaction mixture comprising the sample as follows: (i) lysing red blood cells, (ii) affinity capturing granulocytes, and (iii) separating the captured granulocytes from the reaction mixture. In some embodiments, the step of affinity capturing may by carried out by a granulocyte-specific antibody attached to a solid support, such as magnetic beads, MACS (Miltenyi Biotec, Auburn, Calif.). In other embodiments, granulocytes or their component subtypes may be removed by resetting and/or density gradient centrifugation, e.g. RosetteSep® kits (Stem Cell Technologies, Vancouver, BC, Canada); BD Vacutainer® CPT (Becton Dickinson and Company, Franklin Lakes, N.J.); In accordance with the invention, granulocytes may be removed by separately removing all or only one of any of neutrophils, basophils or eosinophils. Granulocyte-specific antibodies are available commercially and may be conveniently attached directly or indirectly to a solid support using conventional methods, e.g. biotinylating a granulocyte-specific antibody and, after incubation with a sample, capturing conjugates via streptavidinated magnetic beads.

### Amplification of Nucleic Acid Populations

As noted below, amplicons of target populations of nucleic acids may be generated by a variety of amplification techniques. In one aspect of the invention, multiplex PCR is used to amplify members of a mixture of nucleic acids, particularly mixtures comprising recombined immune molecules such as T cell receptors, B cell receptors, or portions thereof. Guidance for carrying out multiplex PCRs of such immune molecules is found in the following references, which are incorporated by reference: Faham et al, U.S. patent publication 2011/0207134; Lim et al, U.S. patent publication 2008/0166718; and the like. As described more fully below, in one aspect, the step of spatially isolating individual nucleic acid molecules is achieved by carrying out a primary multiplex amplification of a preselected somatically rearranged region or portion thereof (i.e. target sequences) using forward and reverse primers that each have tails non-complementary to the target sequences to produce a first amplicon whose member sequences have common sequences at each end that allow further manipulation. For example, such common ends may include primer binding sites for continued amplification using just a single forward primer and a single reverse primer instead of multiples of each, or for bridge amplification of individual molecules on a solid surface, or the like. Such common ends may be added in a single amplification as described above, or they may be added in a two-step procedure to avoid difficulties associated with manufacturing and exercising quality control over mixtures of long primers (e.g. 50-70 bases or more). In such a two-step process (described more fully below), the primary amplification is carried out as described above, except that the primer tails are limited in length to provide only forward and reverse primer binding sites at the ends of the sequences of the first amplicon. A secondary amplification is then carried out using secondary amplification primers specific to these primer binding sites to add further sequences to the ends of a second amplicon. The secondary amplification primers have tails non-complementary to the target sequences, which form the ends of the second amplicon and which may be used in connection with sequencing the clonotypes of the second amplicon. In one embodiment, such added sequences may include primer binding sites for generating sequence reads and primer binding sites for carrying out bridge PCR on a solid surface to generate clonal populations of spatially isolated individual molecules, for example, when Solexa-based sequencing is used. In this latter approach, a sample of sequences from the second amplicon are disposed on a solid surface that has attached complementary oligonucleotides capable of annealing to sequences of the sample, after which cycles of primer extension, denaturation, annealing are implemented until clonal populations of templates are formed. Preferably, the size of the sample is selected so that (i) it includes an effective representation of clonotypes in the original sample, and (ii) the density of clonal populations on the solid surface is in a range that permits unambiguous sequence determination of clonotypes.

The region to be amplified can include the full clonal sequence or a subset of the clonal sequence, including the V-D junction, D-J junction of an immunoglobulin gene, the full variable region of an immunoglobulin, the antigen recognition region, or a CDR, e.g., complementarity determining region 3 (CDR3).

After amplification of DNA from the genome (or amplification of nucleic acid in the form of cDNA by reverse transcribing RNA), the individual nucleic acid molecules can be isolated, optionally re-amplified, and then sequenced individually. Exemplary amplification protocols may be found in van Dongen et al, Leukemia, 17: 2257-2317 (2003) or van Dongen et al, U.S. patent publication 2006/0234234, which is incorporated by reference. Briefly, an exemplary protocol is as follows: Reaction buffer: ABI Buffer II or ABI Gold Buffer (Life Technologies, San Diego, Calif.); 50 μL final reaction volume; 100 ng sample DNA; 10 pmol of each primer (subject to adjustments to balance amplification as described below); dNTPs at 200 μM final concentration; MgCl₂ at 1.5 mM final concentration (subject to optimization depending on target sequences and polymerase); Taq polymerase (1-2 U/tube); cycling conditions: preactivation 7 min at 95° C.; annealing at 60° C.; cycling times: 30 s denaturation; 30 s annealing; 30 s extension. Polymerases that can be used for amplification in the methods of the invention are commercially available and include, for example, Taq polymerase, AccuPrime polymerase, or Pfu. The choice of polymerase to use can be based on whether fidelity or efficiency is preferred.

Methods for isolation of nucleic acids from a pool include subcloning nucleic acid into DNA vectors and transforming bacteria (bacterial cloning), spatial separation of the molecules in two dimensions on a solid substrate (e.g., glass slide), spatial separation of the molecules in three dimensions in a solution within micelles (such as can be achieved using oil emulsions with or without immobilizing the molecules on a solid surface such as beads), or using microreaction chambers in, for example, microfluidic or nano-fluidic chips. Dilution can be used to ensure that on average a single molecule is present in a given volume, spatial region, bead, or reaction chamber. Guidance for such methods of isolating individual nucleic acid molecules is found in the following references: Sambrook, Molecular Cloning: A Laboratory Manual (Cold Spring Harbor Laboratory Press, 2001s); Shendure et al, Science, 309: 1728-1732 (including supplemental material) (2005); U.S. Pat. No. 6,300,070; Bentley et al, Nature, 456: 53-59 (including supplemental material) (2008); U.S. Pat. No. 7,323,305; Matsubara et al, Biosensors & Bioelectronics, 20: 1482-1490 (2005): U.S. Pat. No. 6,753,147; and the like.

Real time PCR, picogreen staining, nanofluidic electrophoresis (e.g. LabChip) or UV absorption measurements can be used in an initial step to judge the functional amount of amplifiable material.

In one aspect, multiplex amplifications are carried out so that relative amounts of sequences in a starting population are substantially the same as those in the amplified population, or amplicon. That is, multiplex amplifications are carried out with minimal amplification bias among member sequences of a sample population. In one embodiment, such relative amounts are substantially the same if each relative amount in an amplicon is within five fold of its value in the starting sample. In another embodiment, such relative amounts are substantially the same if each relative amount in an amplicon is within two fold of its value in the starting sample. As discussed more fully below, amplification bias in PCR may be detected and corrected using conventional techniques so that a set of PCR primers may be selected for a predetermined repertoire that provide unbiased amplification of any sample.

In one embodiment, amplification bias may be avoided by carrying out a two-stage amplification (as described above) wherein a small number of amplification cycles are implemented in a first, or primary, stage using primers having tails non-complementary with the target sequences. The tails include primer binding sites that are added to the ends of the sequences of the primary amplicon so that such sites are used in a second stage amplification using only a single forward primer and a single reverse primer, thereby eliminating a primary cause of amplification bias. In some embodiments, the primary PCR will have a small enough number of cycles (e.g. 5-10) to minimize the differential amplification by the different primers. The secondary amplification is done with one pair of primers and hence the issue of differential amplification is minimal. One percent of the primary PCR is taken directly to the secondary PCR. Thirty-five cycles (equivalent to ˜28 cycles without the 100 fold dilution step) used between the two amplifications were sufficient to show a robust amplification irrespective of whether the breakdown of cycles were: one cycle primary and 34 secondary or 25 primary and 10 secondary. Even though ideally doing only 1 cycle in the primary PCR may decrease the amplification bias, there are other considerations. One aspect of this is representation. This plays a role when the starting input amount is not in excess to the number of reads ultimately obtained. For example, if 1,000,000 reads are obtained and starting with 1,000,000 input molecules then taking only representation from 100,000 molecules to the secondary amplification would degrade the precision of estimating the relative abundance of the different species in the original sample. The 100 fold dilution between the 2 steps means that the representation is reduced unless the primary PCR amplification generated significantly more than 100 molecules. This indicates that a minimum 8 cycles (256 fold), but more comfortably 10 cycle (˜1,000 fold), may be used. The alternative to that is to take more than 1% of the primary PCR into the secondary but because of the high concentration of primer used in the primary PCR, a big dilution factor is can be used to ensure these primers do not interfere in the amplification and worsen the amplification bias between sequences. Another alternative is to add a purification or enzymatic step to eliminate the primers from the primary PCR to allow a smaller dilution of it. In this example, the primary PCR was 10 cycles and the second 25 cycles.

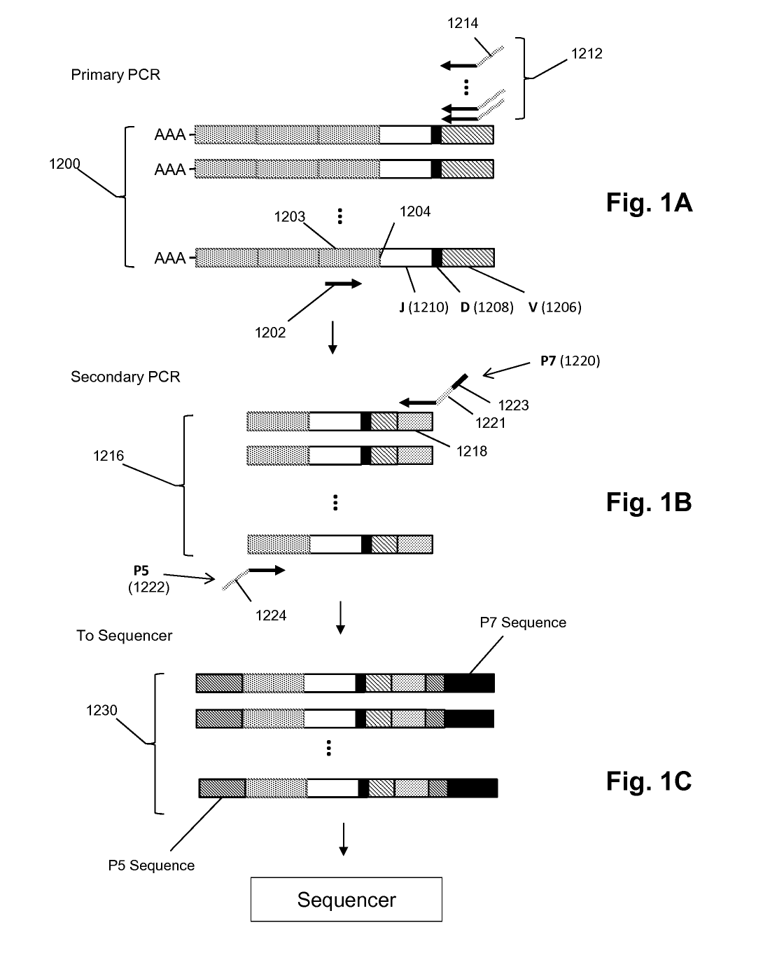

Briefly, the scheme of Faham and Willis (cited above) for amplifying IgH-encoding nucleic acids (RNA) is illustrated in FIGS. 1A-1C. Nucleic acids (1200) are extracted from lymphocytes in a sample and combined in a PCR with a primer (1202) specific for C region (1203) and primers (1212) specific for the various V regions (1206) of the immunoglobulin or TCR genes. Primers (1212) each have an identical tail (1214) that provides a primer binding site for a second stage of amplification. As mentioned above, primer (1202) is positioned adjacent to junction (1204) between the C region (1203) and J region (1210). In the PCR, amplicon (1216) is generated that contains a portion of C-encoding region (1203), J-encoding region (1210), D-encoding region (1208), and a portion of V-encoding region (1206). Amplicon (1216) is further amplified in a second stage using primer P5 (1222) and primer P7 (1220), which each have tails (1224 and 1221/1223, respectively) designed for use in an Illumina DNA sequencer. Tail (1221/1223) of primer P7 (1220) optionally incorporates tag (1221) for labeling separate samples in the sequencing process. Second stage amplification produces amplicon (1230) which may be used in an Illumina DNA sequencer.

### Generating Sequence Reads

Any high-throughput technique for sequencing nucleic acids can be used in the method of the invention. Preferably, such technique has a capability of generating in a cost-effective manner a volume of sequence data from which at least 1000 clonotypes can be determined, and preferably, from which at least 10,000 to 1,000,000 clonotypes can be determined. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. In one aspect of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm². In one embodiment, sequencing chemistries are employed having relatively high error rates. In such embodiments, the average quality scores produced by such chemistries are monotonically declining functions of sequence read lengths. In one embodiment, such decline corresponds to 0.5 percent of sequence reads have at least one error in positions 1-75; 1 percent of sequence reads have at least one error in positions 76-100; and 2 percent of sequence reads have at least one error in positions 101-125.

In one aspect, a sequence-based clonotype profile of an individual is obtained using the following steps: (a) obtaining a nucleic acid sample from B-cells of the individual; (b) spatially isolating individual molecules derived from such nucleic acid sample, the individual molecules comprising at least one template generated from a nucleic acid in the sample, which template comprises a somatically rearranged region or a portion thereof, each individual molecule being capable of producing at least one sequence read; (c) sequencing said spatially isolated individual molecules; and (d) determining abundances of different sequences of the nucleic acid molecules from the nucleic acid sample to generate the clonotype profile. In one embodiment, each of the somatically rearranged regions comprise a V region and a J region. In another embodiment, the step of sequencing comprises bidirectionally sequencing each of the spatially isolated individual molecules to produce at least one forward sequence read and at least one reverse sequence read. Further to the latter embodiment, at least one of the forward sequence reads and at least one of the reverse sequence reads have an overlap region such that bases of such overlap region are determined by a reverse complementary relationship between such sequence reads. In still another embodiment, each of the somatically rearranged regions comprise a V region and a J region and the step of sequencing further includes determining a sequence of each of the individual nucleic acid molecules from one or more of its forward sequence reads and at least one reverse sequence read starting from a position in a J region and extending in the direction of its associated V region. In another embodiment, individual molecules comprise nucleic acids selected from the group consisting of complete IgH molecules, incomplete IgH molecules. In another embodiment, the step of sequencing comprises generating the sequence reads having monotonically decreasing quality scores. Further to the latter embodiment, monotonically decreasing quality scores are such that the sequence reads have error rates no better than the following: 0.2 percent of sequence reads contain at least one error in base positions 1 to 50, 0.2 to 1.0 percent of sequence reads contain at least one error in positions 51-75, 0.5 to 1.5 percent of sequence reads contain at least one error in positions 76-100. In another embodiment, the above method comprises the following steps: (a) obtaining a nucleic acid sample from T-cells and/or B-cells of the individual; (b) spatially isolating individual molecules derived from such nucleic acid sample, the individual molecules comprising nested sets of templates each generated from a nucleic acid in the sample and each containing a somatically rearranged region or a portion thereof, each nested set being capable of producing a plurality of sequence reads each extending in the same direction and each starting from a different position on the nucleic acid from which the nested set was generated; (c) sequencing said spatially isolated individual molecules; and (d) determining abundances of different sequences of the nucleic acid molecules from the nucleic acid sample to generate the clonotype profile. In one embodiment, the step of sequencing includes producing a plurality of sequence reads for each of the nested sets. In another embodiment, each of the somatically rearranged regions comprise a V region and a J region, and each of the plurality of sequence reads starts from a different position in the V region and extends in the direction of its associated J region.

In one aspect, for each sample from an individual, the sequencing technique used in the methods of the invention generates sequences of least 1000 clonotypes per run; in another aspect, such technique generates sequences of at least 10,000 clonotypes per run; in another aspect, such technique generates sequences of at least 100,000 clonotypes per run; in another aspect, such technique generates sequences of at least 500,000 clonotypes per run; and in another aspect, such technique generates sequences of at least 1,000,000 clonotypes per run. In still another aspect, such technique generates sequences of between 100,000 to 1,000,000 clonotypes per run per individual sample. In some embodiments, each of the foregoing numbers of clonotypes are determined from 2 to 100 sequence reads; in other embodiments, each of the foregoing numbers of clonotypes are determined from 2 to 10 sequence reads; in still other embodiments, each of the foregoing numbers of clonotypes are determined from at least 10 sequence reads.

The sequencing technique used in the methods of the provided invention can generate about 30 bp, about 40 bp, about 50 bp, about 60 bp, about 70 bp, about 80 bp, about 90 bp, about 100 bp, about 110, about 120 bp per read, about 150 bp, about 200 bp, about 250 bp, about 300 bp, about 350 bp, about 400 bp, about 450 bp, about 500 bp, about 550 bp, or about 600 bp per read.

### Generating Clonotypes from Sequence Data

Constructing clonotypes from sequence read data is disclosed in Faham and Willis (cited above), which is incorporated herein by reference. Briefly, constructing clonotypes from sequence read data depends in part on the sequencing method used to generate such data, as the different methods have different expected read lengths and data quality. In one approach, a Solexa sequencer is employed to generate sequence read data for analysis. In one embodiment, a sample is obtained that provides at least 0.5-1.0×10⁶ lymphocytes to produce at least 1 million template molecules, which after optional amplification may produce a corresponding one million or more clonal populations of template molecules (or clusters). For most high throughput sequencing approaches, including the Solexa approach, such over sampling at the cluster level is desirable so that each template sequence is determined with a large degree of redundancy to increase the accuracy of sequence determination. For Solexa-based implementations, preferably the sequence of each independent template is determined 10 times or more. For other sequencing approaches with different expected read lengths and data quality, different levels of redundancy may be used for comparable accuracy of sequence determination. Those of ordinary skill in the art recognize that the above parameters, e.g. sample size, redundancy, and the like, are design choices related to particular applications.

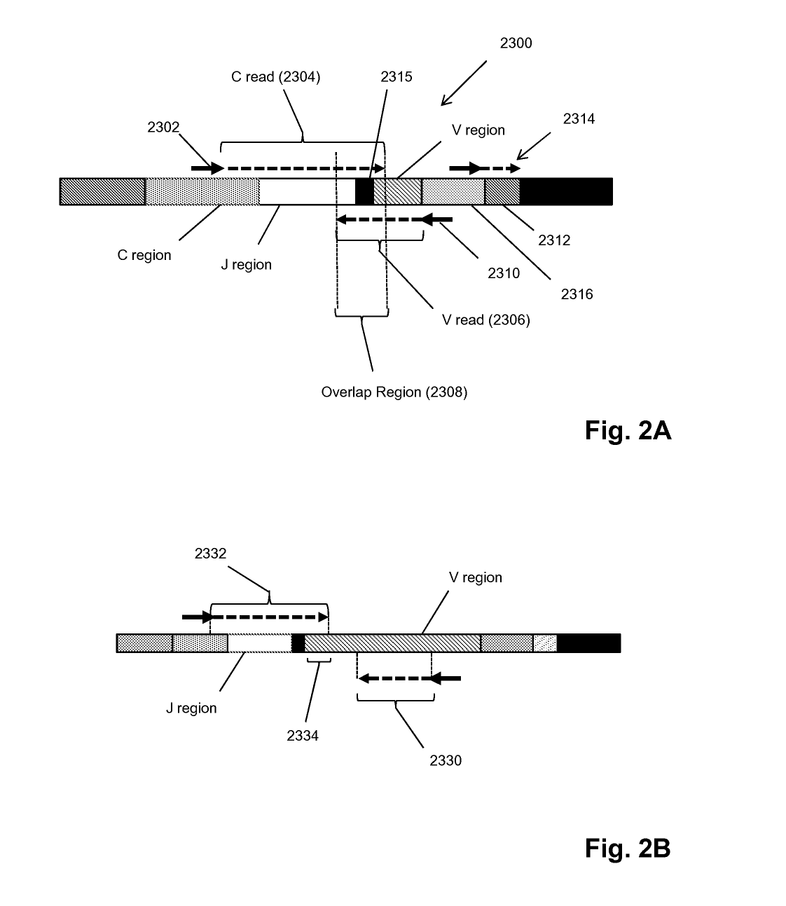

In one aspect, clonotypes of IgH chains (illustrated in FIG. 2A) are determined by at least one sequence read starting in its C region and extending in the direction of its associated V region (referred to herein as a “C read” (2304)) and at least one sequence read starting in its V region and extending in the direction of its associated J region (referred to herein as a “V read” (2306)). Such reads may or may not have an overlap region (2308) and such overlap may or may not encompass the NDN region (2315) as shown in FIG. 2A. Overlap region (2308) may be entirely in the J region, entirely in the NDN region, entirely in the V region, or it may encompass a J region-NDN region boundary or a V region-NDN region boundary, or both such boundaries (as illustrated in FIG. 2A). Typically, such sequence reads are generated by extending sequencing primers, e.g. (2302) and (2310) in FIG. 2A, with a polymerase in a sequencing-by-synthesis reaction, e.g. Metzger, Nature Reviews Genetics, 11: 31-46 (2010); Fuller et al, Nature Biotechnology, 27: 1013-1023 (2009). The binding sites for primers (2302) and (2310) are predetermined, so that they can provide a starting point or anchoring point for initial alignment and analysis of the sequence reads. In one embodiment, a C read is positioned so that it encompasses the D and/or NDN region of the IgH chain and includes a portion of the adjacent V region, e.g. as illustrated in FIGS. 2A and 2B. In one aspect, the overlap of the V read and the C read in the V region is used to align the reads with one another. In other embodiments, such alignment of sequence reads is not necessary, so that a V read may only be long enough to identify the particular V region of a clonotype. This latter aspect is illustrated in FIG. 2B. Sequence read (2330) is used to identify a V region, with or without overlapping another sequence read, and another sequence read (2332) traverses the NDN region and is used to determine the sequence thereof. Portion (2334) of sequence read (2332) that extends into the V region is used to associate the sequence information of sequence read (2332) with that of sequence read (2330) to determine a clonotype. For some sequencing methods, such as base-by-base approaches like the Solexa sequencing method, sequencing run time and reagent costs are reduced by minimizing the number of sequencing cycles in an analysis. Optionally, as illustrated in FIG. 2A, amplicon (2300) is produced with sample tag (2312) to distinguish between clonotypes originating from different biological samples, e.g. different patients. Sample tag (2312) may be identified by annealing a primer to primer binding region (2316) and extending it (2314) to produce a sequence read across tag (2312), from which sample tag (2312) is decoded.

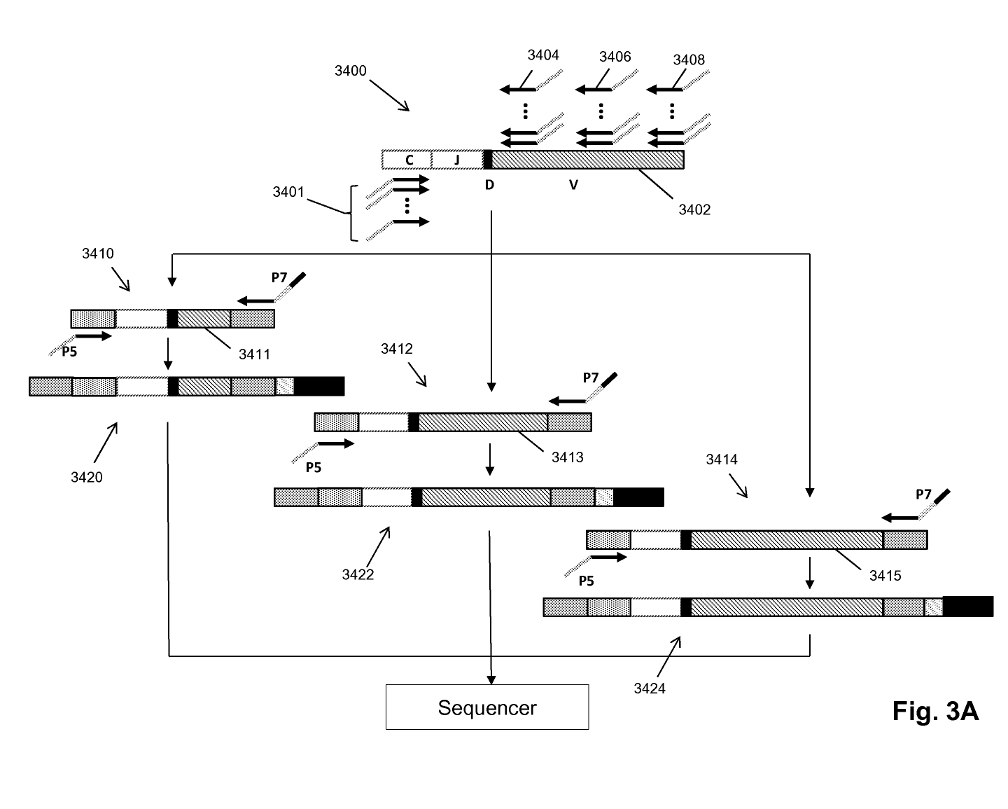

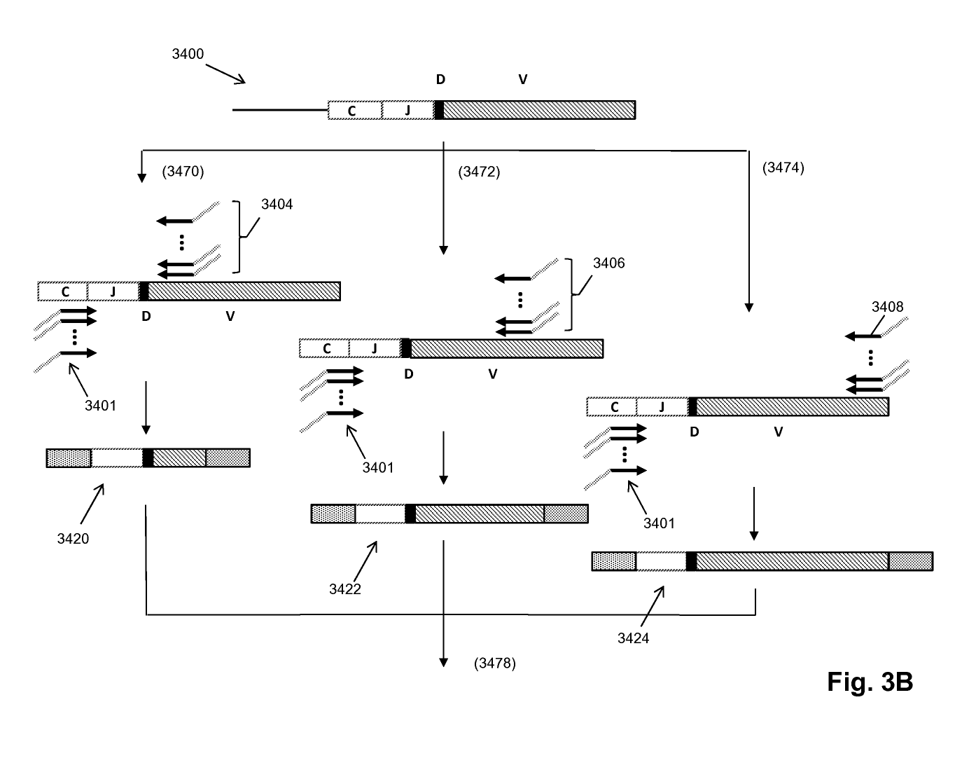

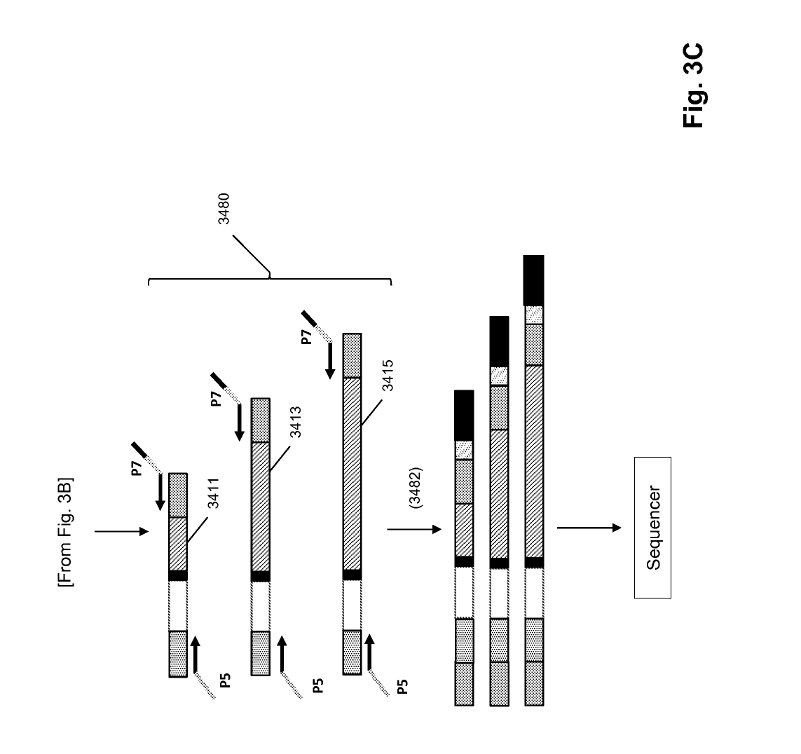

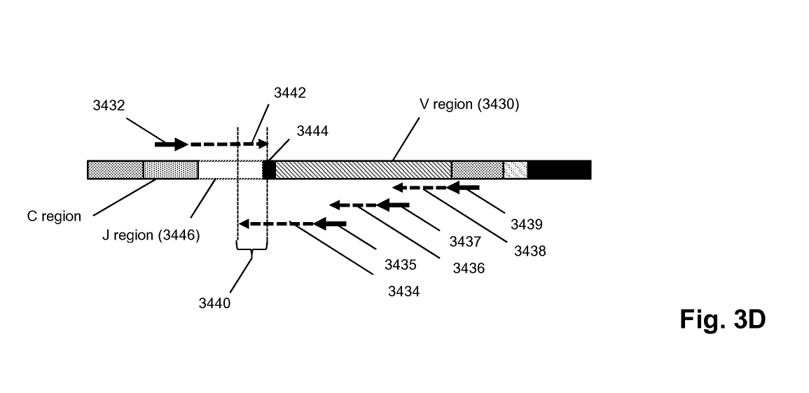

In one aspect of the invention, sequences of clonotypes may be determined by combining information from one or more sequence reads, for example, along the V(D)J regions of the selected chains. In another aspect, sequences of clonotypes are determined by combining information from a plurality of sequence reads. Such pluralities of sequence reads may include one or more sequence reads along a sense strand (i.e. “forward” sequence reads) and one or more sequence reads along its complementary strand (i.e. “reverse” sequence reads). When multiple sequence reads are generated along the same strand, separate templates are first generated by amplifying sample molecules with primers selected for the different positions of the sequence reads. This concept is illustrated in FIG. 3A where primers (3404, 3406 and 3408) are employed to generate amplicons (3410, 3412, and 3414, respectively) in a single reaction. Such amplifications may be carried out in the same reaction or in separate reactions. In one aspect, whenever PCR is employed, separate amplification reactions are used for generating the separate templates which, in turn, are combined and used to generate multiple sequence reads along the same strand. This latter approach is preferable for avoiding the need to balance primer concentrations (and/or other reaction parameters) to ensure equal amplification of the multiple templates (sometimes referred to herein as “balanced amplification” or “unbias amplification”). The generation of templates in separate reactions is illustrated in FIGS. 3B-3C. There a sample containing IgH (3400) is divided into three portions (3470, 3472, and 3474) which are added to separate PCRs using J region primers (3401) and V region primers (3404, 3406, and 3408, respectively) to produce amplicons (3420, 3422 and 3424, respectively). The latter amplicons are then combined (3478) in secondary PCR (3480) using P5 and P7 primers to prepare the templates (3482) for bridge PCR and sequencing on an Illumina GA sequencer, or like instrument.

Sequence reads of the invention may have a wide variety of lengths, depending in part on the sequencing technique being employed. For example, for some techniques, several trade-offs may arise in its implementation, for example, (i) the number and lengths of sequence reads per template and (ii) the cost and duration of a sequencing operation. In one embodiment, sequence reads are in the range of from 20 to 3400 nucleotides; in another embodiment, sequence reads are in a range of from 30 to 200 nucleotides; in still another embodiment, sequence reads are in the range of from 30 to 120 nucleotides. In one embodiment, 1 to 4 sequence reads are generated for determining the sequence of each clonotype; in another embodiment, 2 to 4 sequence reads are generated for determining the sequence of each clonotype; and in another embodiment, 2 to 3 sequence reads are generated for determining the sequence of each clonotype. In the foregoing embodiments, the numbers given are exclusive of sequence reads used to identify samples from different individuals. The lengths of the various sequence reads used in the embodiments described below may also vary based on the information that is sought to be captured by the read; for example, the starting location and length of a sequence read may be designed to provide the length of an NDN region as well as its nucleotide sequence; thus, sequence reads spanning the entire NDN region are selected. In other aspects, one or more sequence reads that in combination (but not separately) encompass a D and/or NDN region are sufficient.

In another aspect of the invention, sequences of clonotypes are determined in part by aligning sequence reads to one or more V region reference sequences and one or more J region reference sequences, and in part by base determination without alignment to reference sequences, such as in the highly variable NDN region. A variety of alignment algorithms may be applied to the sequence reads and reference sequences. For example, guidance for selecting alignment methods is available in Batzoglou, Briefings in Bioinformatics, 6: 6-22 (2005), which is incorporated by reference. In one aspect, whenever V reads or C reads (as mentioned above) are aligned to V and J region reference sequences, a tree search algorithm is employed, e.g. as described generally in Gusfield (cited above) and Cormen et al, Introduction to Algorithms, Third Edition (The MIT Press, 2009).

The construction of IgH clonotypes from sequence reads is characterized by at least two factors: i) the presence of somatic mutations which makes alignment more difficult, and ii) the NDN region is larger so that it is often not possible to map a portion of the V segment to the C read. In one aspect of the invention, this problem is overcome by using a plurality of primer sets for generating V reads, which are located at different locations along the V region, preferably so that the primer binding sites are nonoverlapping and spaced apart, and with at least one primer binding site adjacent to the NDN region, e.g. in one embodiment from 5 to 50 bases from the V-NDN junction, or in another embodiment from 10 to 50 bases from the V-NDN junction. The redundancy of a plurality of primer sets minimizes the risk of failing to detect a clonotype due to a failure of one or two primers having binding sites affected by somatic mutations. In addition, the presence of at least one primer binding site adjacent to the NDN region makes it more likely that a V read will overlap with the C read and hence effectively extend the length of the C read. This allows for the generation of a continuous sequence that spans all sizes of NDN regions and that can also map substantially the entire V and J regions on both sides of the NDN region. Embodiments for carrying out such a scheme are illustrated in FIGS. 3A and 3D. In FIG. 3A, a sample comprising IgH chains (3400) are sequenced by generating a plurality amplicons for each chain by amplifying the chains with a single set of J region primers (3401) and a plurality (three shown) of sets of V region (3402) primers (3404, 3406, 3408) to produce a plurality of nested amplicons (e.g., 3410, 3412, 3414) all comprising the same NDN region and having different lengths encompassing successively larger portions (3411, 3413, 3415) of V region (3402). Members of a nested set may be grouped together after sequencing by noting the identify (or substantial identity) of their respective NDN, J and/or C regions, thereby allowing reconstruction of a longer V(D)J segment than would be the case otherwise for a sequencing platform with limited read length and/or sequence quality. In one embodiment, the plurality of primer sets may be a number in the range of from 2 to 5. In another embodiment the plurality is 2-3; and still another embodiment the plurality is 3. The concentrations and positions of the primers in a plurality may vary widely. Concentrations of the V region primers may or may not be the same. In one embodiment, the primer closest to the NDN region has a higher concentration than the other primers of the plurality, e.g. to insure that amplicons containing the NDN region are represented in the resulting amplicon. In a particular embodiment where a plurality of three primers is employed, a concentration ratio of 60:20:20 is used. One or more primers (e.g. 3435 and 3437 in FIG. 3D) adjacent to the NDN region (3444) may be used to generate one or more sequence reads (e.g. 3434 and 3436) that overlap the sequence read (3442) generated by J region primer (3432), thereby improving the quality of base calls in overlap region (3440). Sequence reads from the plurality of primers may or may not overlap the adjacent downstream primer binding site and/or adjacent downstream sequence read. In one embodiment, sequence reads proximal to the NDN region (e.g. 3436 and 3438) may be used to identify the particular V region associated with the clonotype. Such a plurality of primers reduces the likelihood of incomplete or failed amplification in case one of the primer binding sites is hypermutated during immunoglobulin development. It also increases the likelihood that diversity introduced by hypermutation of the V region will be capture in a clonotype sequence. A secondary PCR may be performed to prepare the nested amplicons for sequencing, e.g. by amplifying with the P5 (3401) and P7 (3404, 3406, 3408) primers as illustrated to produce amplicons (3420, 3422, and 3424), which may be distributed as single molecules on a solid surface, where they are further amplified by bridge PCR, or like technique.

Somatic Hypermutations. In one embodiment, IgH-based clonotypes that have undergone somatic hypermutation are determined as follows. A somatic mutation is defined as a sequenced base that is different from the corresponding base of a reference sequence (of the relevant segment, usually V, J or C) and that is present in a statistically significant number of reads. In one embodiment, C reads may be used to find somatic mutations with respect to the mapped J segment and likewise V reads for the V segment. Only pieces of the C and V reads are used that are either directly mapped to J or V segments or that are inside the clonotype extension up to the NDN boundary. In this way, the NDN region is avoided and the same ‘sequence information’ is not used for mutation finding that was previously used for clonotype determination (to avoid erroneously classifying as mutations nucleotides that are really just different recombined NDN regions). For each segment type, the mapped segment (major allele) is used as a scaffold and all reads are considered which have mapped to this allele during the read mapping phase. Each position of the reference sequences where at least one read has mapped is analyzed for somatic mutations. In one embodiment, the criteria for accepting a non-reference base as a valid mutation include the following: 1) at least N reads with the given mutation base, 2) at least a given fraction N/M reads (where M is the total number of mapped reads at this base position) and 3) a statistical cut based on the binomial distribution, the average Q score of the N reads at the mutation base as well as the number (M-N) of reads with a non-mutation base. Preferably, the above parameters are selected so that the false discovery rate of mutations per clonotype is less than 1 in 1000, and more preferably, less than 1 in 10000.

It is expected that PCR error is concentrated in some bases that were mutated in the early cycles of PCR. Sequencing error is expected to be distributed in many bases even though it is totally random as the error is likely to have some systematic biases. It is assumed that some bases will have sequencing error at a higher rate, say 5% (5 fold the average). Given these assumptions, sequencing error becomes the dominant type of error. Distinguishing PCR errors from the occurrence of highly related clonotypes will play a role in analysis. Given the biological significance to determining that there are two or more highly related clonotypes, a conservative approach to making such calls is taken. The detection of enough of the minor clonotypes so as to be sure with high confidence (say 99.9%) that there are more than one clonotype is considered. For example of clonotypes that are present at 100 copies/1,000,000, the minor variant is detected 14 or more times for it to be designated as an independent clonotype. Similarly, for clonotypes present at 1,000 copies/1,000,000 the minor variant can be detected 74 or more times to be designated as an independent clonotype. This algorithm can be enhanced by using the base quality score that is obtained with each sequenced base. If the relationship between quality score and error rate is validated above, then instead of employing the conservative 5% error rate for all bases, the quality score can be used to decide the number of reads that need to be present to call an independent clonotype. The median quality score of the specific base in all the reads can be used, or more rigorously, the likelihood of being an error can be computed given the quality score of the specific base in each read, and then the probabilities can be combined (assuming independence) to estimate the likely number of sequencing error for that base. As a result, there are different thresholds of rejecting the sequencing error hypothesis for different bases with different quality scores. For example for a clonotype present at 1,000 copies/1,000,000 the minor variant is designated independent when it is detected 22 and 74 times if the probability of error were 0.01 and 0.05, respectively.

In the presence of sequencing errors, each genuine clonotype is surrounded by a ‘cloud’ of reads with varying numbers of errors with respect to the its sequence. The “cloud” of sequencing errors drops off in density as the distance increases from the clonotype in sequence space. A variety of algorithms are available for converting sequence reads into clonotypes. In one aspect, coalescing of sequence reads (that is, merging candidate clonotypes determined to have one or more sequencing errors) depends on at least three factors: the number of sequences obtained for each of the clonotypes being compared; the number of bases at which they differ; and the sequencing quality score at the positions at which they are discordant. A likelihood ratio may be constructed and assessed that is based on the expected error rates and binomial distribution of errors. For example, two clonotypes, one with 150 reads and the other with 2 reads with one difference between them in an area of poor sequencing quality will likely be coalesced as they are likely to be generated by sequencing error. On the other hand two clonotypes, one with 100 reads and the other with 50 reads with two differences between them are not coalesced as they are considered to be unlikely to be generated by sequencing error. In one embodiment of the invention, the algorithm described below may be used for determining clonotypes from sequence reads. In one aspect of the invention, sequence reads are first converted into candidate clonotypes. Such a conversion depends on the sequencing platform employed. For platforms that generate high Q score long sequence reads, the sequence read or a portion thereof may be taken directly as a candidate clonotype. For platforms that generate lower Q score shorter sequence reads, some alignment and assembly steps may be required for converting a set of related sequence reads into a candidate clonotype. For example, for Solexa-based platforms, in some embodiments, candidate clonotypes are generated from collections of paired reads from multiple clusters, e.g. 10 or more, as mentioned above

The cloud of sequence reads surrounding each candidate clonotype can be modeled using the binomial distribution and a simple model for the probability of a single base error. This latter error model can be inferred from mapping V and J segments or from the clonotype finding algorithm itself, via self-consistency and convergence. A model is constructed for the probability of a given ‘cloud’ sequence Y with read count C2 and E errors (with respect to sequence X) being part of a true clonotype sequence X with perfect read count C1 under the null model that X is the only true clonotype in this region of sequence space. A decision is made whether or not to coalesce sequence Y into the clonotype X according the parameters C1, C2, and E. For any given C1 and E a max value C2 is pre-calculated for deciding to coalesce the sequence Y. The max values for C2 are chosen so that the probability of failing to coalesce Y under the null hypothesis that Y is part of clonotype X is less than some value P after integrating over all possible sequences Y with error E in the neighborhood of sequence X. The value P is controls the behavior of the algorithm and makes the coalescing more or less permissive.

If a sequence Y is not coalesced into clonotype X because its read count is above the threshold C2 for coalescing into clonotype X then it becomes a candidate for seeding separate clonotypes. An algorithm implementing such principles makes sure that any other sequences Y2, Y3, etc. which are ‘nearer’ to this sequence Y (that had been deemed independent of X) are not aggregated into X. This concept of ‘nearness’ includes both error counts with respect to Y and X and the absolute read count of X and Y, i.e. it is modeled in the same fashion as the above model for the cloud of error sequences around clonotype X. In this way ‘cloud’ sequences can be properly attributed to their correct clonotype if they happen to be ‘near’ more than one clonotype.

In one embodiment, an algorithm proceeds in a top down fashion by starting with the sequence X with the highest read count. This sequence seeds the first clonotype. Neighboring sequences are either coalesced into this clonotype if their counts are below the precalculated thresholds (see above), or left alone if they are above the threshold or ‘closer’ to another sequence that was not coalesced. After searching all neighboring sequences within a maximum error count, the process of coalescing reads into clonotype X is finished. Its reads and all reads that have been coalesced into it are accounted for and removed from the list of reads available for making other clonotypes. The next sequence is then moved on to with the highest read count. Neighboring reads are coalesced into this clonotype as above and this process is continued until there are no more sequences with read counts above a given threshold, e.g. until all sequences with more than 1 count have been used as seeds for clonotypes.

As mentioned above, in another embodiment of the above algorithm, a further test may be added for determining whether to coalesce a candidate sequence Y into an existing clonotype X, which takes into account quality score of the relevant sequence reads. The average quality score(s) are determined for sequence(s) Y (averaged across all reads with sequence Y) were sequences Y and X differ. If the average score is above a predetermined value then it is more likely that the difference indicates a truly different clonotype that should not be coalesced and if the average score is below such predetermined value then it is more likely that sequence Y is caused by sequencing errors and therefore should be coalesced into X.

### Clans of Related Clonotypes

Frequently lymphocytes produce related clonotypes. That is, multiple lymphocytes may exist or develop that produce clonotypes whose sequences are similar. This may be due to a variety of mechanism, such as hypermutation in the case of IgH molecules. As another example, in cancers, such as lymphoid neoplasms, a single lymphocyte progenitor may give rise to many related lymphocyte progeny, each possessing and/or expressing a slightly different TCR or BCR, and therefore a different clonotype, due to cancer-related somatic mutation(s), such as base substitutions, aberrant rearrangements, or the like. A set of such related clonotypes is referred to herein as a “clan.” In some case, clonotypes of a clan may arise from the mutation of another clan member. Such an “offspring” clonotype may be referred to as a phylogenic clonotype. Clonotypes within a clan may be identified by one or more measures of relatedness to a parent clonotype, or to each other. In one embodiment, clonotypes may be grouped into the same clan by percent homology, as described more fully below. In another embodiment, clonotypes may be assigned to a clan by common usage of V regions, J regions, and/or NDN regions. For example, a clan may be defined by clonotypes having common J and ND regions but different V regions; or it may be defined by clonotypes having the same V and J regions (including identical base substitutions mutations) but with different NDN regions; or it may be defined by a clonotype that has undergone one or more insertions and/or deletions of from 1-10 bases, or from 1-5 bases, or from 1-3 bases, to generate clan members. In another embodiment, members of a clan are determined as follows.

Clonotypes are assigned to the same clan if they satisfy the following criteria: i) they are mapped to the same V and J reference segments, with the mappings occurring at the same relative positions in the clonotype sequence, and ii) their NDN regions are substantially identical. “Substantial” in reference to clan membership means that some small differences in the NDN region are allowed because somatic mutations may have occurred in this region. Preferably, in one embodiment, to avoid falsely calling a mutation in the NDN region, whether a base substitution is accepted as a cancer-related mutation depends directly on the size of the NDN region of the clan. For example, a method may accept a clonotype as a clan member if it has a one-base difference from clan NDN sequence(s) as a cancer-related mutation if the length of the clan NDN sequence(s) is m nucleotides or greater, e.g. 9 nucleotides or greater, otherwise it is not accepted, or if it has a two-base difference from clan NDN sequence(s) as cancer-related mutations if the length of the clan NDN sequence(s) is n nucleotides or greater, e.g. 20 nucleotides or greater, otherwise it is not accepted, In another embodiment, members of a clan are determined using the following criteria: (a) V read maps to the same V region, (b) C read maps to the same J region, (c) NDN region substantially identical (as described above), and (d) position of NDN region between V-NDN boundary and J-NDN boundary is the same (or equivalently, the number of downstream base additions to D and the number of upstream base additions to D are the same). Clonotypes of a single sample may be grouped into clans and clans from successive samples acquired at different times may be compared with one another. In particular, in one aspect of the invention, clans containing clonotypes correlated with a disease, such as a lymphoid neoplasm, are identified from clonotypes of each sample and compared with that of the immediately previous sample to determine disease status, such as, continued remission, incipient relapse, evidence of further clonal evolution, or the like. As used herein, “size” in reference to a clan means the number of clonotypes in the clan.

As mentioned above, in one aspect, methods of the invention monitor a level of a clan of clonotypes rather than an individual clonotype. This is because of the phenomena of clonal evolution, e.g. Campbell et al, Proc. Natl. Acad. Sci., 105: 13081-13086 (2008); Gerlinger et al, Br. J. Cancer, 103: 1139-1143 (2010). The sequence of a clone that is present in the diagnostic sample may not remain exactly the same as the one in a later sample, such as one taken upon a relapse of disease. Therefore if one is following the exact clonotype sequence that matches the diagnostic sample sequence, the detection of a relapse might fail. Such evolved clone are readily detected and identified by sequencing. For example many of the evolved clones emerge by V region replacement (called VH replacement). These types of evolved clones are missed by real time PCR techniques since the primers target the wrong V segment. However given that the D-J junction stays intact in the evolved clone, it can be detected and identified in this invention using the sequencing of individual spatially isolated molecules. Furthermore, the presence of these related clonotypes at appreciable frequency in the diagnostic sample increases the likelihood of the relevance of the clonotype. Similarly the development of somatic hypermutations in the immune receptor sequence may interfere with the real time PCR probe detection, but appropriate algorithms applied to the sequencing readout (as disclosed above) can still recognize a clonotype as an evolving clonotype. For example, somatic hypermutations in the V or J segments can be recognized. This is done by mapping the clonotypes to the closest germ line V and J sequences. Differences from the germ line sequences can be attributed to somatic hypermutations. Therefore clonotypes that evolve through somatic hypermutations in the V or J segments can be readily detected and identified. Somatic hypermutations in the NDN region can be predicted. When the remaining D segment is long enough to be recognized and mapped, any somatic mutation in it can be readily recognized. Somatic hypermutations in the N+P bases (or in D segment that is not mappable) cannot be recognized for certain as these sequences can be modified in newly recombined cells which may not be progeny of the cancerous clonotype. However algorithms are readily constructed to identify base changes that have a high likelihood of being due to somatic mutation. For example a clonotype with the same V and J segments and 1 base difference in the NDN region from the original clone(s) has a high likelihood of being the result of somatic recombination. This likelihood can be increased if there are other somatic hypermutations in the V and J segments because this identifies this specific clonotype as one that has been the subject of somatic hypermutation. Therefore the likelihood of a clonotype being the result of somatic hypermutation from an original clonotype can be computed using several parameters: the number of differences in the NDN region, the length of NDN region, as well as the presence of other somatic hypermutations in the V and/or J segments.

The clonal evolution data can be informative. For example if the major clone is an evolved clone (one that was absent previously, and therefore, previously unrecorded) then this is an indication of that tumor has acquired new genetic changes with potential selective advantages. This is not to say that the specific changes in the immune cell receptor are the cause of the selective advantage but rather that they may represent a marker for it. Tumors whose clonotypes have evolved can potentially be associated with differential prognosis. In one aspect of the invention, a clonotype or clonotypes being used as a patient-specific biomarker of a disease, such as a lymphoid neoplasm, for example, a leukemia, includes previously unrecorded clonotypes that are somatic mutants of the clonotype or clonotypes being monitored. In another aspect, whenever any previously unrecorded clonotype is at least ninety percent homologous to an existing clonotype or group of clonotypes serving as patient-specific biomarkers, then such homologous clonotype is included with or in the group of clonotypes being monitored going forward. That is, if one or more patient-specific clonotypes are identified in a lymphoid neoplasm and used to periodically monitor the disease (for example, by making measurement on less invasively acquired blood samples) and if in the course of one such measurement a new (previously unrecorded) clonotype is detected that is a somatic mutation of a clonotype of the current set, then it is added to the set of patient-specific clonotypes that are monitored for subsequent measurements. In one embodiment, if such previously unrecorded clonotype is at least ninety percent homologous with a member of the current set, then it is added to the patient-specific set of clonotype biomarkers for the next test carried out on the patient; that is, the such previously unrecorded clonotype is included in the clan of the member of the current set of clonotypes from which it was derived (based on the above analysis of the clonotype data). In another embodiment, such inclusion is carried out if the previously unrecorded clonotype is at least ninety-five percent homologous with a member of the current set. In another embodiment, such inclusion is carried out if the previously unrecorded clonotype is at least ninety-eight percent homologous with a member of the current set.

It is also possible that a cell evolves through a process that replaces the NDN region but preserves the V and J segment along with their accumulated mutations. Such cells can be identified as previously unrecorded cancer clonotypes by the identification of the common V and J segment provided they contain a sufficient number of mutations to render the chance of these mutations being independently derived small. A further constraint may be that the NDN region is of similar size to the previously sequenced clone.

### Example

A phase 1/2 study of immunotransplant for newly diagnosed MCL patients was initiated to test the hypothesis that immunotransplant will amplify anti-tumor T cells. Anti-tumor T cells are assessed by co-culturing autologous tumor with peripheral blood T cells and measuring their production of: IFNg, TNF, IL2, CD137, perforin and granzyme by multiplex surface and intracellular flow cytometry. A secondary endpoint is measurement of molecular residual disease (MRD) using allele-specific oligonucleotide PCR (ASO PCR) and immune receptor repertoire analysis, which employs consensus primers for universal amplification and sequencing of nucleic acids encoding immunoglobulins. The study is powered to detect a 50% improvement in sustained molecular remission rate compared to recent trials of standard transplant, e.g. Pott et al, Blood, 115(16): 3215-3223 (2010); Geisler et al, Blood, 112(7): 2687-2693 (2008). Thirteen of twenty-five enrolled patients completed the immunotransplant protocol. 83% of patients showed amplification of antitumor T cells post-immunotransplant. We have observed differing patterns of antitumor T cells among these patients, with some having CD8 T cell responses, CD4 T cell responses, or a combination of the two. TCRβ clonotypes specific to the vaccination were identified by TCRβ repertoire analysis. MRD testing using the sequence-based BCR analysis in blood samples was positive immediately post-transplant in 3 patients, 2 of which ultimately relapsed and expired. In these cases, relapse was detected by the method of the invention 14 months and 4.5 months prior to detection by PET or MRI imaging techniques, respectively. In the patient that relapsed at 4.5 months, evaluation of the bone marrow using standard methods was negative, and the relapse was thought to be restricted to the CNS. 77% of patients (10/13) were MRD negative throughout the first year after transplant. Molecular relapse was seen in 15% of patients (2/13) after 1 year, and these patients are being followed for clinical relapse. Pre-clinically, amplification of anti-tumor T cells correlates with cure of even myeloablation-resistant disease. The reiteration of anti-tumor T cell amplification in our preliminary patient data suggests that immunotransplant may improve clinical outcomes. Immune repertoire sequencing by the method of the invention provided an effective measurement of MRD bypassing the assay individualization of ASO PCR and has been shown to be more sensitive than conventional PET and MRI.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### DEFINITIONS

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Aligning” means a method of comparing a test sequence, such as a sequence read, to one or more reference sequences to determine which reference sequence or which portion of a reference sequence is closest based on some sequence distance measure. An exemplary method of aligning nucleotide sequences is the Smith Waterman algorithm. Distance measures may include Hamming distance, Levenshtein distance, or the like. Distance measures may include a component related to the quality values of nucleotides of the sequences being compared.

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Clonality” as used herein means a measure of the degree to which the distribution of clonotype abundances among clonotypes of a repertoire is skewed to a single or a few clonotypes. Roughly, clonality is an inverse measure of clonotype diversity. Many measures or statistics are available from ecology describing species-abundance relationships that may be used for clonality measures in accordance with the invention, e.g. Chapters 17 & 18, in Pielou, An Introduction to Mathematical Ecology, (Wiley-Interscience, 1969). In one aspect, a clonality measure used with the invention is a function of a clonotype profile (that is, the number of distinct clonotypes detected and their abundances), so that after a clonotype profile is measured, clonality may be computed from it to give a single number. One clonality measure is Simpson's measure, which is simply the probability that two randomly drawn clonotypes will be the same. Other clonality measures include information-based measures and McIntosh's diversity index, disclosed in Pielou (cited above).

“Clonotype” means a recombined nucleotide sequence of a T cell or B cell encoding a T cell receptor (TCR) or B cell receptor (BCR), or a portion thereof. In one aspect, a collection of all the distinct clonotypes of a population of lymphocytes of an individual is a repertoire of such population, e.g. Arstila et al, Science, 286: 958-961 (1999); Yassai et al, Immunogenetics, 61: 493-502 (2009); Kedzierska et al, Mol. Immunol., 45(3): 607-618 (2008); and the like. As used herein, “clonotype profile,” or “repertoire profile,” is a tabulation of clonotypes of a sample of T cells and/or B cells (such as a peripheral blood sample containing such cells) that includes substantially all of the repertoire's clonotypes and their relative abundances. “Clonotype profile,” “repertoire profile,” and “repertoire” are used herein interchangeably. (That is, the term “repertoire,” as discussed more fully below, means a repertoire measured from a sample of lymphocytes). In one aspect of the invention, clonotypes comprise portions of an immunoglobulin heavy chain (IgH) or a TCR β chain. In other aspects of the invention, clonotypes may be based on other recombined molecules, such as immunoglobulin light chains or TCRα chains, or portions thereof.

“Coalescing” means treating two candidate clonotypes with sequence differences as the same by determining that such differences are due to experimental or measurement error and not due to genuine biological differences. In one aspect, a sequence of a higher frequency candidate clonotype is compared to that of a lower frequency candidate clonotype and if predetermined criteria are satisfied then the number of lower frequency candidate clonotypes is added to that of the higher frequency candidate clonotype and the lower frequency candidate clonotype is thereafter disregarded. That is, the read counts associated with the lower frequency candidate clonotype are added to those of the higher frequency candidate clonotype.

“Complementarity determining regions” (CDRs) mean regions of an immunoglobulin (i.e., antibody) or T cell receptor where the molecule complements an antigen's conformation, thereby determining the molecule's specificity and contact with a specific antigen. T cell receptors and immunoglobulins each have three CDRs: CDR1 and CDR2 are found in the variable (V) domain, and CDR3 includes some of V, all of diverse (D) (heavy chains only) and joint (J), and some of the constant (C) domains.

“Lymphoid neoplasm” means an abnormal proliferation of lymphocytes that may be malignant or non-malignant. A lymphoid cancer is a malignant lymphoid neoplasm. Lymphoid neoplasms are the result of, or are associated with, lymphoproliferative disorders, including but not limited to, follicular lymphoma, chronic lymphocytic leukemia (CLL), acute lymphocytic leukemia (ALL), hairy cell leukemia, lymphomas, multiple myeloma, post-transplant lymphoproliferative disorder, mantle cell lymphoma (MCL), diffuse large B cell lymphoma (DLBCL), T cell lymphoma, or the like, e.g. Jaffe et al, Blood, 112: 4384-4399 (2008); Swerdlow et al, WHO Classification of Tumours of Haematopoietic and Lymphoid Tissues (e. 4^{th}) (IARC Press, 2008).

“Minimal residual disease” means remaining cancer cells after treatment. The term is most frequently used in connection with treatment of lymphomas and leukemias.

“Percent homologous,” “percent identical,” or like terms used in reference to the comparison of a reference sequence and another sequence (“comparison sequence”) mean that in an optimal alignment between the two sequences, the comparison sequence is identical to the reference sequence in a number of subunit positions equivalent to the indicated percentage, the subunits being nucleotides for polynucleotide comparisons or amino acids for polypeptide comparisons. As used herein, an “optimal alignment” of sequences being compared is one that maximizes matches between subunits and minimizes the number of gaps employed in constructing an alignment. Percent identities may be determined with commercially available implementations of algorithms, such as that described by Needleman and Wunsch, J. Mol. Biol., 48: 443-453 (1970) (“GAP” program of Wisconsin Sequence Analysis Package, Genetics Computer Group, Madison, Wis.), or the like. Other software packages in the art for constructing alignments and calculating percentage identity or other measures of similarity include the “BestFit” program, based on the algorithm of Smith and Waterman, Advances in Applied Mathematics, 2: 482-489 (1981) (Wisconsin Sequence Analysis Package, Genetics Computer Group, Madison, Wis.). In other words, for example, to obtain a polynucleotide having a nucleotide sequence at least 95 percent identical to a reference nucleotide sequence, up to five percent of the nucleotides in the reference sequence may be deleted or substituted with another nucleotide, or a number of nucleotides up to five percent of the total number of nucleotides in the reference sequence may be inserted into the reference sequence.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Quality score” means a measure of the probability that a base assignment at a particular sequence location is correct. A variety methods are well known to those of ordinary skill for calculating quality scores for particular circumstances, such as, for bases called as a result of different sequencing chemistries, detection systems, base-calling algorithms, and so on. Generally, quality score values are monotonically related to probabilities of correct base calling. For example, a quality score, or Q, of 10 may mean that there is a 90 percent chance that a base is called correctly, a Q of 20 may mean that there is a 99 percent chance that a base is called correctly, and so on. For some sequencing platforms, particularly those using sequencing-by-synthesis chemistries, average quality scores decrease as a function of sequence read length, so that quality scores at the beginning of a sequence read are higher than those at the end of a sequence read, such declines being due to phenomena such as incomplete extensions, carry forward extensions, loss of template, loss of polymerase, capping failures, deprotection failures, and the like.

“Repertoire”, or “immune repertoire”, as used herein means a set of distinct recombined nucleotide sequences that encode B cell receptors (BCRs), or fragments thereof, respectively, in a population of lymphocytes of an individual, wherein the nucleotide sequences of the set have a one-to-one correspondence with distinct lymphocytes or their clonal subpopulations for substantially all of the lymphocytes of the population. In one aspect, a population of lymphocytes from which a repertoire is determined is taken from one or more tissue samples, such as one or more blood samples. A member nucleotide sequence of a repertoire is referred to herein as a “clonotype.” In one aspect, clonotypes of a repertoire comprises any segment of nucleic acid common to a B cell population which has undergone somatic recombination during the development of BCRs, including normal or aberrant (e.g. associated with cancers) precursor molecules thereof, including, but not limited to, any of the following: an immunoglobulin heavy chain (IgH) or subsets thereof (e.g. an IgH variable region, CDR3 region, or the like), incomplete IgH molecules, an immunoglobulin light chain or subsets thereof (e.g. a variable region, CDR region, or the like), a CDR (including CDR1, CDR2 or CDR3, of BCRs, or combinations of such CDRs), V(D)J regions of BCRs, hypermutated regions of IgH variable regions, or the like. In one aspect, nucleic acid segments defining clonotypes of a repertoire are selected so that their diversity (i.e. the number of distinct nucleic acid sequences in the set) is large enough so that substantially every B cell or clone thereof in an individual carries a unique nucleic acid sequence of such repertoire. That is, in accordance with the invention, a practitioner may select for defining clonotypes a particular segment or region of recombined nucleic acids that encode BCRs that do not reflect the full diversity of a population of B cells; however, preferably, clonotypes are defined so that they do reflect the diversity of the population of B cells from which they are derived. That is, preferably each different clone of a sample has different clonotype. (Of course, in some applications, there will be multiple copies of one or more particular clonotypes within a profile, such as in the case of samples from leukemia or lymphoma patients). In other aspects of the invention, the population of lymphocytes corresponding to a repertoire may be circulating B cells, or other subpopulations defined by cell surface markers, or the like. Such subpopulations may be acquired by taking samples from particular tissues, e.g. bone marrow, or lymph nodes, or the like, or by sorting or enriching cells from a sample (such as peripheral blood) based on one or more cell surface markers, size, morphology, or the like. In still other aspects, the population of lymphocytes corresponding to a repertoire may be derived from disease tissues, such as a tumor tissue, an infected tissue, or the like. In one embodiment, a repertoire comprising human IgH chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶. In a particular embodiment, a repertoire of the invention comprises a set of nucleotide sequences encoding substantially all segments of the V(D)J region of an IgH chain. In one aspect, “substantially all” as used herein means every segment having a relative abundance of 0.001 percent or higher; or in another aspect, “substantially all” as used herein means every segment having a relative abundance of 0.0001 percent or higher. In another particular embodiment, a repertoire of the invention comprises a set of nucleotide sequences that encodes substantially all segments of the V(D)J region of a TCR β chain. In another embodiment, a repertoire of the invention comprises a set of nucleotide sequences having lengths in the range of from 25-200 nucleotides and including segments of the V, D, and J regions of an IgH chain. In another embodiment, a repertoire of the invention comprises a number of distinct nucleotide sequences that is substantially equivalent to the number of lymphocytes expressing a distinct IgH chain. In still another embodiment, “substantially equivalent” means that with ninety-nine percent probability a repertoire of nucleotide sequences will include a nucleotide sequence encoding an IgH or portion thereof carried or expressed by every lymphocyte of a population of an individual at a frequency of 0.001 percent or greater. In still another embodiment, “substantially equivalent” means that with ninety-nine percent probability a repertoire of nucleotide sequences will include a nucleotide sequence encoding an IgH or portion thereof carried or expressed by every lymphocyte present at a frequency of 0.0001 percent or greater. The sets of clonotypes described in the foregoing two sentences are sometimes referred to herein as representing the “full repertoire” of IgH sequences. As mentioned above, when measuring or generating a clonotype profile (or repertoire profile), a sufficiently large sample of lymphocytes is obtained so that such profile provides a reasonably accurate representation of a repertoire for a particular application. In one aspect, samples comprising from 10⁵ to 10⁷ lymphocytes are employed, especially when obtained from peripheral blood samples of from 1-10 mL.

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

What is claimed is:

1. A method of monitoring a mantle cell lymphoma residual disease in a patient by one or more patient-specific clonotypes correlated with the mantle cell lymphoma, the method comprising the steps of:
(a) treating a patient by immunotransplanting the patient with vaccine-primed autologous T cells;
(b) obtaining a peripheral blood sample from the patient comprising B-cells and/or cell-free nucleic acids;
(c) amplifying molecules of nucleic acid from the B-cells of the sample and/or cell-free nucleic acids in the sample, the molecules of nucleic acid comprising recombined DNA sequences from immunoglobulin genes;
(d) sequencing the amplified molecules of nucleic acid to form a clonotype profile; and
(e) determining from the clonotype profile a presence, absence and/or level of the one or more patient-specific clonotypes correlated with the mantle cell lymphoma, including phylogenic clonotypes thereof.

2. The method of claim 1 further including the step of repeating said steps (b) through (e) to monitor said mantle cell lymphoma residual disease in the patient.

3. The method of claim 1 wherein each of said clonotype profiles includes every clonotype present at a frequency of 0.01 percent or greater with a probability of ninety-nine percent.

4. The method of claim 1 wherein each of said clonotype profiles includes at least 10⁴ clonotypes.

5. The method of claim 1 wherein said one or more patient-specific clonotypes correlated with said mantle cell lymphoma are determined from a bone marrow sample or a lymph node sample of the patient.

6. A method of monitoring a mantle cell lymphoma in a patient by one or more patient-specific clonotypes correlated with the mantle cell lymphoma, the method comprising the steps of:
(a) treating a patient by immunotransplanting the patient with vaccine-primed autologous T cells;
(b) obtaining a peripheral blood sample from the patient comprising B-cells, plasma cells and/or cell-free nucleic acids from B-cells and/or plasma cells;
(c) extracting a nucleic acid sample from the peripheral blood sample;
(d) amplifying in a polymerase chain reaction molecules of nucleic acid from nucleic acid sample, the molecules of nucleic acid comprising a VDJ rearrangement of IgH;
(e) sequencing the amplified molecules of nucleic acid to form a clonotype profile; and
(f) determining from the clonotype profile a level of each of the one or more patient-specific clonotypes correlated with the mantle cell lymphoma, wherein such levels include phylogenic clonotypes of each of such one or more patient-specific clonotypes.

7. The method of claim 6 wherein said step of sequencing includes generating sequence reads in a range of from 20 to 400 nucleotides for determining a sequence of each clonotype.

8. The method of claim 6 wherein said step of determining includes correlating a decreased likelihood of remission with said level of said one or more patient-specific clonotypes correlated with said mantle cell lymphoma.
